(19) United States
(12) Patent Application Publication
(10) Pub. No.: US 2021/0030381 A1
(43) Pub. Date: Feb. 4, 2021

(54) IMAGING DEVICE, PROCESS OF MANUFACTURING SUCH A DEVICE AND VISUALIZATION METHOD

(71) Applicant: UNIVERSITÄT BASEL, Basel (CH)

(72) Inventors: Uri NAHUM, Riehen (CH); Carlo SEPPI, Basel (CH); Peter VON NIEDERHÄUSERN, Köniz (CH); Simon PEZOLD, Weil am Rhein (DE); Stephan HAERLE, Meggen (CH); Philippe CATTIN, Windisch (CH)

(21) Appl. No.: 16/964,363

(22) PCT Filed: Jan. 25, 2019
(86) PCT No.: PCT/EP2019/051828

§ 371 Date: Jul. 23, 2020

(30) Foreign Application Priority Data
Jan. 25, 2018 (CH) 00089/18

(51) Int. Cl. A61B 6/00; H04N 5/32

(52) U.S. Cl. CPC A61B 6/4258; G01T 1/164; H04N 5/32; A61B 6/462

(57) ABSTRACT

An imaging device for visualizing a radioactive tracer in a human or animal body (6) comprises: a collimator plate (11) having a plurality of pinholes (111); a radiation detector (2) being arranged adjacent to a detector surface (112) of the collimator plate (11) such that radioactive radiation passing at least one of the plurality of pinholes (111) is received by the radiation detector (2); and an image processing unit (3) adapted to evaluate radiation signals obtained by the radiation detector (2) to determine a three dimensional position of at least one radiation source (61) emitting the radioactive radiation and causing the radiation signals.

### TECHNICAL FIELD

The present invention relates to an imaging device having a collimator and a radiation detector arranged adjacent to the collimator such that radioactive radiation passing the collimator is received by the detector. Such imaging devices can be used for visualizing a radioactive tracer in a human or animal body.

### BACKGROUND ART

In many medical treatments or applications tracers are used for identifying or visualizing items or processes within human or animal bodies. Such tracers often are radioactive substances which are administered, e.g. orally or injected with a syringe, to the human or animal patient and which have properties to suitably behave in the body of the patient such that conclusions related to the medical conditions of the patient can be drawn. Since the substances are radioactive they can be located from outside the body by appropriate means.

For example, for treating tumor patients particularly having tumors in the area of the face, neck or breast it often is important to analyze a sentinel node. Sentinels are the first lymph nodes in the lymphatic systems after the tumor. I.e., sentinels are the lymph nodes neighboring the tumors. Analyzing the sentinel allows for concluding if and to what extent lymph nodes have to be removed for preventing the tumor to propagate.

For locating the tracers within the bodies it is known to use gamma cameras. Such cameras usually have a collimator and a gamma photon detector. The collimator is arranged adjacent to the body where the tracer is suspected. Gamma photons which are emitted by the tracer and which permeate the body are provided through the collimator and are detected by the gamma photon detector. The gamma photon detector provides signals which precisely correspond to the emission of gamma photons by the tracer.

However, since such gamma cameras only detect gamma photons it usually is quite difficult to find the exact original position of the tracers in the patient. Particularly, whereas such cameras may allow for evaluating from which direction the tracer emits the radiation, conclusion as the exact position of the tracer is often not sufficiently accurate and reliable.

Therefore, there is a need for a device or process allowing a precise and reliable detection of a tracer or of its distribution in a human or animal body in an efficient way and, particularly, allowing an efficient and precise detection of a sentinel node.

### DISCLOSURE OF THE INVENTION

According to the invention this need is settled by an imaging device as it is defined by the features of independent claim 1, by a method as it is defined by the features of independent claim 18, and by a process as it is defined by the features of independent claim 33. Preferred embodiments are subject of the dependent claims.

In one aspect, the invention is an imaging device for visualizing a radioactive tracer in a human or animal body. The imaging device comprises a collimator plate having a plurality of pinholes, a radiation detector and an image processing unit. The radiation detector is arranged adjacent to a detector surface of the collimator plate such that radioactive radiation passing at least one of the plurality of pinholes is received by the detector. The image processing unit is adapted to evaluate radiation signals obtained by the detector to determine a three dimensional position of at least one radiation source emitting the radioactive radiation and causing the radiation signals.

The image processing unit can be or comprise a computer or computing device. Such computer or device may have any combination of a central processing unit (CPU), a random access memory (RAM), a read only memory (ROM) and a data storage as well as additional elements.

In order to be adapted in accordance with the invention, the image processing unit can be programmed. Thereby, it can be switched or circuited appropriately for being hardware programmed. Or, it can run or execute an application for being software programmed. Also, combinations of hardware and software programming are possible.

The term “adjacent” as used in connection with the detector and the collimator plate can relate to an arrangement in which radiation passing the pinholes essentially reaches the detector in an unhindered manner. Thereby, the collimator plate may be in contact with the detector or not.

The term “radioactive tracer” as used in connection with the invention relates to a typically chemical compound in which one or more atoms are radioisotopes. By virtue of its radioactive decay the radioactive tracer can be used to explore the mechanism of chemical reactions by tracing the path that the radioisotope follows from reactants to products. In particular, radioactive tracers can be specific to react with a particular tissue in order to accumulate or stay there.

The radioactive radiation can be gamma radiation. In such embodiments, the radiation detector can be a gamma photon detector. The collimator plate and the detector of the imaging device can form or be comprised by a gamma sensor or collimator gamma sensor.

The collimator plate can be any essentially three dimensional and advantageously flat structure appropriate to prevent or essentially restrict the radioactive radiation to pass. Only where the pinholes are located, the radioactive radiation can pass the plate. Preferably, the collimator plate is a single piece or monolithic structure. It can be made of a material such as lead or the like.

The collimator plate can be comprised by a collimator or collimator unit having elements other than the collimator plate. The pinholes can be embodied as bores provided through the collimator plate. The term “plate” as used in connection with the collimator plate can relate to a flat three dimensional structure. Typically, such plates have even or flat top and bottom surfaces. Further, they usually have top and side surfaces which are considerably larger than the side surfaces.

By having the collimator plate with multiple pinholes, it can be achieved that the at least one radiation source such as a lymph node or sentinel is captured from different angles. Due to these different angles also plural radiation sources can be mapped on different three dimensional positions. This parallax effect can be readily used to estimate the distance of the radiation source from the collimator. It even might allow for differentiating two radiation sources that are behind each other and as such indistinguishable from each other with known imaging devices.

The term “radiation signal” relates to any suitable signal indicative of the radiation arriving at the detector. Thereby, a radiation signal can be a specific pattern of current induced in a conductive structure. Such pattern can be composed of current in a characterizing sequence and/or amperage. Or, the radiation signal can be a data packet, advantageously in a predefined structure such as according to a data protocol. Each data signal can be indicative for the location where the radiation hits the detector and/or for strength of the radiation on the detector.

The imaging device allows for efficiently localizing the radiation source(s) which can be essential for taking appropriate measures. For example, the imaging device can be positioned in proximity of a human or animal body or patient to which a tracer is provided and which might be appropriately prepared. The imaging device then provides the information about the three dimensional position of the radiation source(s) and an operator or practitioner can perform a suitable intervention.

Since the imaging device is equipped with the image processing unit, the device can be embodied comparably simple. For example, it can be sufficient that a collimator having the collimator plate is a simple construction of a suitable radiation absorbing material which is equipped with the pinholes, e.g. in the form of simple through bores.

Thus, the imaging device according to the invention allows for a precise and reliable detection of a tracer in a human or animal body in an efficient way and, also, for an efficient and precise detection of a sentinel.

The imaging device can be embodied to provide the determined three dimensional position of the radiation source(s) in any suitable manner. For example, it can have means for generating acoustic and/or tactile signals allowing the user of the device to know where exactly the respective radiation source is.

Preferably, the imaging device comprises a display, wherein the image processing unit is adapted to show the three dimensional position of the radiation source(s) on the display. Such a display can be appropriate and beneficial to precisely inform a user or operator about the three dimensional position of the radiation source(s) such as a cancerous lymph nodes, sentinels or the like.

The term “cancerous lymph node” as used herein relates to a lymph node having cancerous tissue. Such cancerous tissue can be caused by a tumor connected to the lymph node via the lymphatic system.

Thereby, the image processing unit preferably is adapted to show the three dimensional position of the at least one radiation source on the display in real-time. Like this, the imaging device can provide assistance live during a specific action such as, e.g., during a surgical intervention for removing the sentinel lymph nodes and/or other lymph nodes.

In one preferred embodiment, the display comprises a transparent structure which is positionable such that the human or animal body is visible though the transparent structure. The transparent structure can be a glass plate or window. Preferably, the display comprises eyeglasses having a frame holding a lens as the transparent structure of the display. The eyeglasses can be embodied as augmented reality (AR) glasses which do augment the real situation with the information about the three dimensional position of the at least one radiation source. For example, an operator can wear the eyeglasses during intervention wherein his view on the patient is constantly augmented with information about the three dimensional position of the at least one radiation source and other helpful information.

In another preferred embodiment, the imaging device comprises a visual light camera arranged to provide a three dimensional image of at least a section of the human or animal body, wherein the image processing unit is adapted to show the three dimensional position of the radiation source(s) on the three dimensional image of the visual light camera on the display. Like this, the image or movie provided by the visual light camera can be augmented with information about the at least one radiation source. In particular, from the generally non-visible radiation a visual representation can be generated and displayed to a user or operator.

Preferably, the collimator plate is made of a material essentially impervious for the radioactive radiation. In this connection, the term “impervious” can relate to non-penetratable for the radioactive radiation. Thereby, a minor portion of the radiation can still travel directly or be scattered through the plate but a major portion is blocked from penetration.

Preferably, the image processing unit is adapted to calculate probabilities of possible three dimensional positions of the radiation source(s). Like this, the position can be sufficient accurately determined at a comparably high speed. Particularly, in the context of identifying a sentinel or sentinel lymph node such a determination can be appropriate. Thereby, the image processing unit preferably is adapted to select a possible three dimensional position having the highest probability of the possible three dimensional positions as the three dimensional position of the radiation source(s).

Additionally or alternatively, the image processing unit can be adapted to calculate at least one angle based on the radiation signals obtained by the detector which are induced by the radioactive radiation passing different pinholes of the collimator plate for determining the three dimensional position of the at least one radiation source. Since there is a plurality of pinholes provided the radiation source(s) can provide radiation through plural pinholes, wherein the angle between the photons hitting the detector can be indicative for the distance to the detector and the relative position thereto. Like this, a comparably precise determination of the three dimensional position of the radiation source(s) is possible.

Again additionally or alternatively, the image processing unit can be adapted to evaluate radiation intensities based on the radiation signals obtained by the detector which are induced by the radioactive radiation passing different pinholes of the collimator plate for determining the three dimensional position of the at least one radiation source. Such intensities can be used for further enhancing the accuracy of the determination of the three dimensional position.

Preferably, the image processing unit is adapted to provide a graphical representation reproducing the at least one radiation source at their three dimensional position. In particular, the graphical representation can comprise a graphical representation data signal. Such a signal can cause a display to show the graphical representation.

Thereby, the image processing unit preferably is adapted to prepare the radiation signals by applying image processing when evaluating the radiation signals obtained by the detector. Such image processing preferably comprises any combination of denoising such as total variation denoising and filtering such as Gauss-filtering. By applying image processing the quality of the determination of the three dimensional position of the at least one radiation source can be enhanced. In particular, disturbances as they may occur due to radiation scattering and radiation passing the collimator plate besides the pinholes can be removed or minimized.

Preferably, the radiation detector is arranged adjacent to the detector surface of the collimator plate such that radioactive radiation passing the pinholes of the collimator plate unimpededly propagates to the detector surface of the detector. Like this, it can be prevented that septum walls forming passages or compartments are provided to the collimator plate such that a simpler setup and a better evaluation of the detected radiation can be achieved. The collimator plate can further have an exposure surface opposite the detector surface and the exposure surface can be unimpededly exposable to the radioactive radiation of the at least one radiation source. In particular, the complete exposure surface can be unimpededly exposable to the radioactive radiation. Thereby, the plurality of pinholes can extend straightly from the exposure surface to the detector surface through the collimator plate. If the collimator plate is integrated in a collimator or collimator unit a box structure can be arranged between the collimator plate and the detector. In a simple embodiment, the box structure consists of or comprises side walls which form an interior extending from the detector surface and being open towards the detector. The side walls can be made of a material impervious for the radioactive radiation of the at least one radiation source. For example they can be made of the same material as the collimator plate.

Preferably, the imaging device comprises a geometric calibration structure stationary to the collimator plate and the image processing unit is adapted to determine a position of the collimator plate with respect to the detector by means of the geometric calibration structure. The geometric calibration structure can be any predefined geometric form such as rectangular or triangular elements which allow for determining position and orientation of the collimator plate. Such geometric structure allows for an efficient and accurate calibration of the imaging device.

Thereby, the geometric calibration structure preferably comprises three geometric elements. Such a number of elements allow for an efficient and precise calibration. Also, the calibration structure can be arranged in one plane.

Preferably, the plurality of pinholes is non-symmetrically distributed in the collimator plate. Like this and by not having any septum walls defining compartments of passages, an improved depth estimation of the at least one radiation source is possible. Particularly, it has been shown that compared to a regular or symmetric distribution better results can be achieved.

Preferably, the collimator plate comprises a number of the pinholes per square centimeter, the number being approximately 1 or approximately 2.

In a further aspect, the invention is a method of visualizing a sentinel lymph node of a human or animal patient. The method comprises: (i) administering a radioactive tracer to the patient; (ii) positioning an imaging device according to any one of the preceding claims in proximity of the patient, preferably, to be directed to a face, neck or breast of the patient; (iii) obtaining radiation signals caused by at least one radiation source emitting radioactive radiation which is induced by the radioactive tracer wherein, preferably the radiation signals are provided by a detector of the imaging device; (iv) evaluating the detected radiation signals; (v) determining a three dimensional position of the at least one radiation source on the basis of the evaluated radiation signals; and (vi) displaying the three dimensional position to a user.

When being administered, the radioactive tracer at its target location can form a radiation source propagating a radioactive radiation. In some instances, it can take some time for the tracer to be at its specific target location such that it has to be waited, e.g. for a couple of hours, before the image device can be applied. In order to provide the radiation signals, the detector can be positioned in a field of radiation propagation of the at least one radiation source.

Such methods and their preferred embodiments described below allow for implementing the effects end benefits described above in connection with the imaging device and its preferred embodiments in a sentinel analysis application. This enables an efficient evaluation of the conditions of the body with respect to a tumor such as to decide how far the lymphatic system is influenced by the tumor.

The three dimensional position of the at least one radiation source can be determined by an image processing unit of the imaging device evaluating the radiation signals. Preferably, the method comprises a step of overlaying signals of a visible light camera with the determined three dimensional position of the at least one radiation source.

The three dimensional position of the at least one radiation source preferably is displayed in real-time. Further, it preferably is displayed on a transparent structure which is positioned such that the human or animal body is visible though the transparent structure. Such transparent structure preferably is a lens of eyeglasses.

Preferably, determining the three dimensional position of the at least one radiation source comprises calculating probabilities of possible three dimensional positions of the at least one radiation source. Thereby, determining the three dimensional position of the at least one radiation source preferably comprises the step of selecting a possible three dimensional position having the highest probability of the possible three dimensional positions as the three dimensional position of the at least one radiation source. Such calculation allows for efficiently determining the three dimensional position of the at least one radiation source.

Determining the three dimensional position of the at least one radiation source can comprise calculating at least one angle based on the radiation signals obtained by the detector of the imaging device which are induced by the radioactive radiation passing different pinholes of a collimator plate of the imaging device. Further it can comprise evaluating radiation intensities based on the radiation signals obtained by the detector of the imaging device which are induced by the radioactive radiation passing different pinholes of a collimator plate of the imaging device.

Preferably, displaying the three dimensional position to a user comprises providing a graphical representation reproducing the at least one radiation source at its three dimensional position. Such graphical representation can be a symbol, e.g. provided as a symbol signal. Thereby, the symbol signal can be of a similar kind as the radiation signal described above.

Displaying the three dimensional position to a user preferably comprises a step of preparing the radiation signals by applying image processing when evaluating the radiation signals obtained by the detector of the imaging device. Thereby, the image processing preferably comprises any combination of denoising and filtering.

Preferably, the method comprises a step of determining a position of a collimator plate of the imaging device with respect to the detector of the imaging device by means of a geometric calibration structure stationary to the collimator plate.

A collimator plate of the imaging device can have an exposure surface opposite to a detector surface and the exposure surface is unimpededly exposed to the radioactive radiation of the at least one radiation source.

In another further aspect, the invention is a process of manufacturing an imaging device for visualizing a radioactive tracer in a human or animal body. The process comprises: (a) obtaining a collimator plate having a plurality of pinholes; (b) arranging a radiation detector adjacent to a detector surface of the collimator plate such that radioactive radiation passing at least one of the plurality of pinholes is received by the detector; (c) adapting an image processing unit to evaluate radiation signals obtained by the detector to determine a three dimensional position of at least one radiation source emitting the radioactive radiation and causing the radiation signals; and (d) assembling the collimator plate, the detector and the image processing unit.

Such a process and its preferred embodiments described below allow for efficiently manufacturing an imaging device as described above. Thereby, the effects end benefits described above in connection with the imaging device and its preferred embodiments can be achieved.

Preferably, the process comprises obtaining a display and adapting the image processing unit to show the three dimensional position of the at least one radiation source on the display. Thereby, the image processing unit preferably is adapted to show the three dimensional position of the at least one radiation source on the display in real-time.

Preferably, the display comprises a transparent structure which is positionable such that the human or animal body is visible though the transparent structure. Thereby, the display preferably comprises eyeglasses having a frame holding a lens as the transparent structure of the display.

Preferably, the process comprises obtaining a visual light camera; arranging the visual light camera to provide a three dimensional image of at least a section of the human or animal body; and adapting the image processing unit to show the three dimensional position of the at least one radiation source on the three dimensional image of the visual light camera on the display.

The collimator plate preferably is made of a material essentially impervious for the radioactive radiation.

Preferably, the process comprises a step of adapting the image processing unit to calculate probabilities of possible three dimensional positions of the at least one radiation source. Thereby, it preferably further comprises adapting the image processing unit to select a possible three dimensional position having the highest probability of the possible three dimensional positions as the three dimensional position of the at least one radiation source.

The image processing unit can be adapted to calculate at least one angle or distance based on the radiation signals obtained by the detector which are induced by the radioactive radiation passing different pinholes of the collimator plate for determining the three dimensional position of the at least one radiation source.

It can further be adapted to to evaluate radiation intensities based on the radiation signals obtained by the detector which are induced by the radioactive radiation passing different pinholes of the collimator plate for determining the three dimensional position of the at least one radiation source.

Preferably, the process comprises a step of adapting the image processing unit to provide a graphical representation reproducing the at least one radiation source at their three dimensional positions.

Preferably, the process comprises a step of adapting the image processing unit to prepare the radiation signals by applying image processing when evaluating the radiation signals obtained by the detector. Thereby, the image processing preferably comprises any combination of denoising and filtering. The process preferably further comprises providing the collimator plate with an exposure surface opposite the detector surface, wherein the exposure surface is unimpededly exposable to the radioactive radiation of the at least one radiation source.

The process preferably further comprises a step of providing a geometric calibration structure stationary to the collimator plate and adapting the image processing unit to determine a position of the collimator plate with respect to the detector by means of the geometric calibration structure. Thereby, the geometric calibration structure preferably comprises three geometric elements.

Preferably, the process comprises non-symmetrically distributing the plurality of pinholes in the collimator plate. It further preferably comprises equipping the collimator plate with a number of pinholes per square centimeter, the number being about 1 or about 2.

### BRIEF DESCRIPTION OF THE DRAWINGS

The imaging device, the visualization method and the process of manufacture according to the invention are described in more detail below by way of an exemplary embodiment and with reference to the attached drawings, in which:

FIG. 1 shows a perspective view of a collimator of an embodiment of an imaging device according to the invention;

FIG. 2 shows a front view of the collimator of FIG. 1; and

FIG. 3 shows the imaging device of FIG. 1 in operation.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

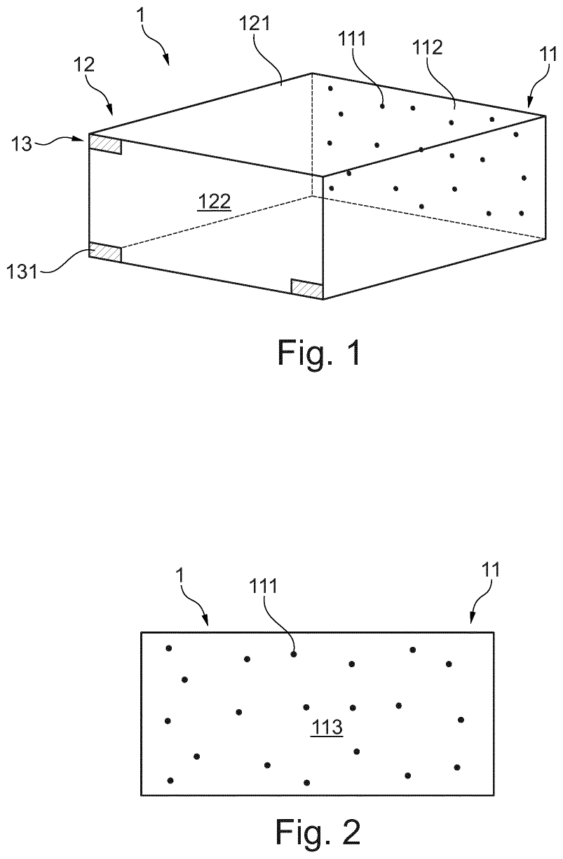

FIG. 1 shows a collimator 1 of an embodiment of an imaging device according to the invention. It comprises a rectangular collimator plate 11 and collimator box 12 as box-like structure. The collimator plate 11 has a detector surface 112 and a plurality of pinholes 111. The collimator box 12 has four rectangular sidewalls 121. It extends from the detector surface 112 of the collimator plate 11 and has an open end 122 opposite to the collimator plate 11. In the Figs. the side walls 121 are transparently depicted in order to allow seeing the interior or the collimator box 12. Typically, the sidewalls 121 are in fact not transparent.

As can be best seen in FIG. 2, the pinholes 111 are non-symmetrically distributed in the collimator plate 11. They can form an irregular pattern on an exposure surface 113 of the collimator plate 11 which pattern can be random or calculated by a suitable algorithm. The pinholes 111 are provided as bores straightly extending from the exposure surface 113 to the detector surface 112 through the collimator plate 11.

Turning back to FIG. 1, the collimator 1 further is equipped with a geometric calibration structure 13 which comprises three rectangles 131. Each of the rectangles is positioned in one angle of the open end 122 of the collimator box 12.

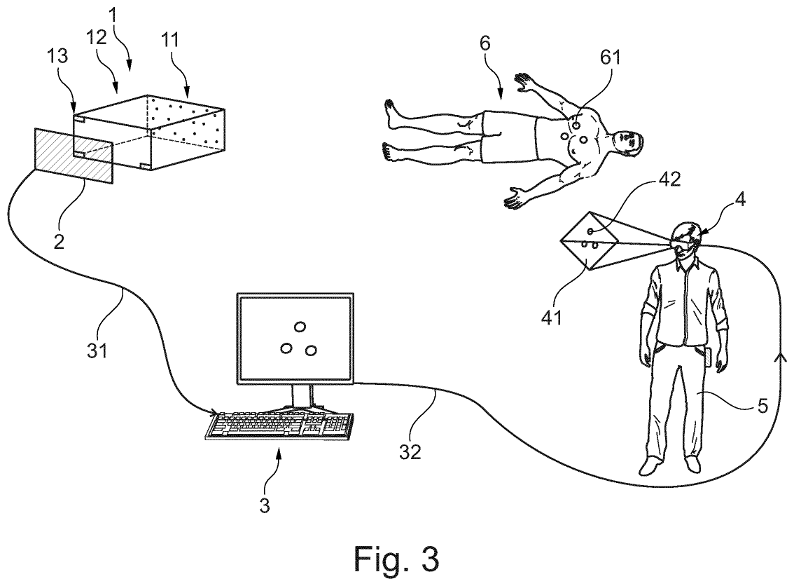

In FIG. 3 the imaging device is shown in operation. Besides the collimator 1 it comprises a detector 2, a computer 3 as image processing unit and augmented reality eyeglasses (AR glasses) 4 as display. The detector 2 has a generally rectangular shape and is positioned adjacent to the collimator box 12 of the collimator 1. In particular, it faces the open end 122 of the collimator box 12 such that radiation passing the pinholes 111 of the collimator plate 11 and escaping the open end 122 of the collimator box 12 unhinderedly reaches the detector 2. Thus, the detector 2 is unimpededly exposed to the radiation traveling through the collimator 1.

In one particular example, the detector 2 is a gamma detector with a resolution of 487×195 pixels, where each pixel is the size of 172 μm×172 μm. The detector 2 has a density of 19.25 g/cm³ Tungsten in the dimensions of 86.9 mm×36 mm×36 mm.

The computer 3 is a desktop computer comprising a central processing unit (CPU), a random access memory (RAM), a read only memory (ROM), a hard disk as data storage, a monitor, a keyboard, a plurality of wired and wireless hardware interfaces such as a local area network (LAN) adapter, a wireless local area network adapter (WLAN), a Bluetooth module, an universal serial bus (USB) and the like, and a mouse. The computer 3 is connected to the detector 2 by a detector interface 31 and to the AR glasses by a AR glasses interface 32. The detector interface 31 and the AR glasses interface 32 are embodied in a suitable wired or wireless manner.

The imaging device is embodied to be used for visualizing a sentinel lymph node of a human patient 6. Thereby, a radioactive tracer is administered to the patient 6. The tracer is then drained through the lymphatic system in particular in the lymph nodes 61 of the patient 6. The first lymph node 61 after the tumor can then be recognized as the one with the highest radioactive radiation. This lymph node is then excised and checked for cancerous tissue. If cancerous tissue is present all lymph nodes in the vicinity are removed, if not, no further lymph nodes are recised.

Then, the imaging device is positioned in proximity of the patient 6 by arranging the collimator 1 together with the detector 2 at the patient 6 and particularly at the patient 6 where the lymph nodes 61 are assumed. The radioactive radiation in the lymph nodes 61 passes the pinholes 111 of the collimator plate 11 and passes through the collimator box 12 to the detector 2. The detector 2 provides radiation signals which are transferred to the computer 3 via the detector interface 31.

The computer 3 runs a computer program or software. The software adapts the computer to evaluate the radiation signals provided by the detector 2 to determine a three dimensional position or distribution of the radioactive tracer in the lymph nodes 61.

In more detail, for preparing the imaging device by calibration, the computer 3 is adapted by the software to determine a position of the collimator plate 11 with respect to the detector 2 by means of the rectangles 131 being stationary to the collimator plate 11. After being calibrated in this way, the computer 3 evaluates the radiation signals by calculating probabilities of possible three dimensional positions or distributions of the tracer in the lymph nodes 61. When evaluating the radiation signals, the computer 3 prepares them or the results of the probabilities calculation by applying image processing. In particular, denoising and filtering is performed by the computer 3. As a further step of the evaluation of the radiation signals, the computer 3 selects three dimensional positions having the highest probability of the real possible three dimensional positions of the lymph nodes 61.

The computer then provides graphical representations 42 reproducing the tracer distribution in the lymph nodes 61 at their three dimensional positions. It transfers graphical representation data signals corresponding to the graphical representations 42 of the lymph nodes 61 to the AR glasses 4 via the AR glasses interface 32.

A practitioner 5 or surgeon carries the AR glasses 4. The AR glasses 4 have a transparent lens. Through the lens, the practitioner sees the patient 6 wherein the AR glasses 4 provide the graphical representations 42 on the lens. Like this, the practitioner 5 sees an augmented view 41 of the patient 6.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfill the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively.

The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

A computer program may be stored/distributed on a suitable medium, such as an optical storage medium or a solid-state medium supplied together with or as part of other hardware, but may also be distributed in other forms, such as via the Internet or other wired or wireless telecommunication systems. In particular, e.g., a computer program can be a computer program product stored on a computer readable medium which computer program product can have computer executable program code adapted to be executed to implement a specific method such as the visualization method according to the invention. Furthermore, a computer program can also be a data structure product or a signal for embodying a specific method such as the method according to the invention.

1.-47. (canceled)

48. An imaging device for visualizing a radioactive tracer in a human or animal body, comprising:
a collimator plate having a plurality of pinholes;
a radiation detector being arranged adjacent to a detector surface of the collimator plate such that radioactive radiation passing at least one of the plurality of pinholes is received by the radiation detector; and
an image processing unit adapted to evaluate radiation signals obtained by the radiation detector to determine a three dimensional position of at least one radiation source emitting the radioactive radiation and causing the radiation signals.

49. The imaging device of claim 48, comprising a display, wherein the image processing unit is adapted to show the three dimensional position of the at least one radiation source on the display, wherein the image processing unit preferably is adapted to show the three dimensional position of the at least one radiation source on the display in real-time.

50. The imaging device of claim 49, wherein the display comprises a transparent structure which is positionable such that the human or animal body is visible though the transparent structure, and wherein the display preferably comprises eyeglasses having a frame holding a lens as the transparent structure of the display.

51. The imaging device of claim 49, comprising a visual light camera arranged to provide a three dimensional image of at least a section of the human or animal body, wherein the image processing unit is adapted to show the three dimensional position of the at least one radiation source on the three dimensional image of the visual light camera on the display.

52. The imaging device of claim 48, wherein the image processing unit is adapted to calculate probabilities of possible three dimensional positions of the at least one radiation source.

53. The imaging device of claim 48, wherein the image processing unit is adapted to provide a graphical representation reproducing the at least one radiation source at its three dimensional position, wherein the image processing unit preferably is adapted to prepare the radiation signals by applying image processing when evaluating the radiation signals obtained by the radiation detector, and wherein the image processing preferably comprises any combination of denoising and filtering.

54. The imaging device of claim 48, wherein the radiation detector is arranged adjacent to the detector surface of the collimator plate such that radioactive radiation passing the pinholes of the collimator plate unimpededly propagates to the radiation detector.

55. The imaging device of claim 48, comprising a geometric calibration structure stationary to the collimator plate and the image processing unit is adapted to determine a position of the collimator plate with respect to the radiation detector by means of the calibration structure, wherein the geometric calibration structure preferably comprises three geometric elements.

56. The imaging device of claim 48, wherein the plurality of pinholes is non-symmetrically distributed in the collimator plate.

57. The imaging device of claim 48, wherein the collimator plate comprises a number of the pinholes per square centimeter, the number being about 1 or about 2, and/or the collimator plate is monolithic, and/or the collimator plate is made of a material essentially impervious for the radioactive radiation.

58. A method of visualizing a sentinel lymph node of a human or animal patient, comprising:
administering a radioactive tracer to the patient;
positioning an imaging device according to claim 48 in proximity of the patient, preferably, to be directed to a face, neck or breast of the patient;
obtaining radiation signals caused by at least one radiation source emitting radioactive radiation which is induced by the radioactive tracer;
evaluating the detected radiation signals;
determining a three dimensional position of the at least one radiation source on the basis of the evaluated radiation signals; and
displaying the three dimensional position of the at least one radiation source to a user, preferably in real-time and/or, preferably, on a transparent structure which is positioned such that the human or animal body is visible though the transparent structure, wherein the transparent structure preferably is a lens of eyeglasses.

59. The method of claim 58, wherein the radiation signals are provided by a radiation detector of the imaging device.

60. The method of claim 58, wherein the radiation signals are evaluated by an image processing unit of the imaging device and the three dimensional position of the at least one radiation source is determined by the image processing unit of the imaging device.

61. The method of claim 58, further comprising overlaying signals of a visible light camera with the determined three dimensional position of the at least one radiation source.

62. The method of claim 58, wherein determining the three dimensional position of the at least one radiation source comprises calculating probabilities of possible three dimensional positions of the at least one radiation source.

63. The method of claim 58, wherein displaying the three dimensional position to a user comprises
providing a graphical representation reproducing the at least one radiation source at its three dimensional position, and/or
preparing the radiation signals by applying image processing when evaluating the radiation signals obtained by the radiation detector of the imaging device, wherein the image processing preferably comprises any combination of denoising and filtering.

64. The method of claim 58, further comprising determining a position of a collimator plate of the imaging device with respect to the radiation detector of the imaging device by means of a geometric calibration structure stationary to the collimator plate.

65. The method of claim 58, wherein a collimator plate of the imaging device has an exposure surface opposite a detector surface and the exposure surface is unimpededly exposed to the radioactive radiation of the at least one radiation source.

66. A process of manufacturing an imaging device for visualizing a radioactive tracer in a human or animal body, comprising:
obtaining a preferably monolithic collimator plate having a plurality of pinholes and, preferably, made of a material essentially impervious for the radioactive radiation;
arranging a radiation detector adjacent to a detector surface of the collimator plate such that radioactive radiation passing at least one of the plurality of pinholes is received by the radiation detector;
adapting an image processing unit to evaluate radiation signals obtained by the radiation detector to determine a three dimensional position of at least one radiation source emitting the radioactive radiation and causing the radiation signals; and
assembling the collimator plate, the radiation detector and the image processing unit.

67. The process of claim 66, further comprising:
obtaining a display and adapting the image processing unit to show the three dimensional position of the at least one radiation source on the display,
wherein the image processing unit is adapted to show the three dimensional position of the at least one radiation source on the display in real-time, and/or
wherein the display comprises a transparent structure which is positionable such that the human or animal body is visible though the transparent structure, wherein the display preferably comprises eyeglasses having a frame holding a lens as the transparent structure of the display.

68. The process of claim 66, further comprising:
obtaining a visual light camera, arranging the visual light camera to provide a three dimensional image of at least a section of the human or animal body, and adapting the image processing unit to show the three dimensional position of the at least one radiation source on the three dimensional image of the visual light camera on the display; and/or
adapting the image processing unit to calculate probabilities of possible three dimensional positions of the at least one radiation source; and/or
adapting the image processing unit to provide a graphical representation reproducing the at least one radiation source at its three dimensional position; and/or
adapting the image processing unit to prepare the radiation signals by applying image processing when evaluating the radiation signals obtained by the radiation detector, wherein the image processing preferably comprises any combination of denoising and filtering; and/or
providing the collimator plate with an exposure surface opposite the detector surface, wherein the exposure surface is unimpededly exposable to the radioactive radiation of the at least one radiation source; and/or
providing a geometric calibration structure stationary to the collimator plate and adapting the image processing unit to determine a position of the collimator plate with respect to the radiation detector, wherein the geometric calibration structure preferably comprises three geometric elements; and/or
non-symmetrically distributing the plurality of pinholes in the collimator plate; and/or
equipping the collimator plate with a number of pinholes per square centimeter, the number being at least 2, or in a range of 2 to about 20, or in a range of about 5 to about 10.
